(19) United States
(12) Patent Application Publication
(10) Pub. No.: US 2007/0276308 A1
(43) Pub. Date: Nov. 29, 2007

(54) Hemostatic agents and devices for the delivery thereof

(76) Inventors: Raymond J. Huey, Orange, CT (US); Denny Lo, Bethlehem, CT (US); Daniel Burns, Stratford, CT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 11/633,687

(22) Filed: Dec. 4, 2006

Related U.S. Application Data
(60) Provisional application No. 60/808,618, filed on May 26, 2006.
(60) Provisional application No. 60/810,447, filed on Jun. 1, 2006.

(51) Int. Cl. A61F 15/00

(52) U.S. Cl. 602/42

(57) ABSTRACT

A hemostatic agent comprises diatomaceous earth in particle form. Devices for promoting hemostasis comprise diatomaceous earth in particle form and a receptacle for retaining the particles therein. The receptacle is defined by a mesh having openings therein. A hemostatic sponge comprises a substrate, diatomaceous earth disposed on the substrate, and a release agent disposed on the substrate. A hemostatic sponge may also comprise a film into which diatomaceous earth is incorporated, or it may comprise a substrate, diatomaceous earth disposed on the substrate, and a film disposed over the diatomaceous earth. A hemostatic sponge may also comprise a first substrate, diatomaceous earth disposed on the first substrate, and a second substrate disposed on the diatomaceous earth. When treating a bleeding wound using any of the foregoing devices, application of the device causes the diatomaceous earth to come into contact with blood to cause a clotting effect.

### CROSS REFERENCE TO RELATED APPLICATIONS

This application is related to U.S. Provisional Patent Application Ser. No. 60/808,618, filed May 26, 2006, entitled “Blood Clotting Compound”; U.S. Provisional Patent Application Ser. No. 60/810,447, filed Jun. 1, 2006, entitled “Hemostatic Device with Oxidized Cellulose Pad”; U.S. patent application Attorney Docket No. 6989-0067, filed Oct. 6, 2006, entitled “Hemostatic Compositions and Method of Manufacture”; U.S. patent application Attorney Docket No. 6989-0069, filed Oct. 20, 2006, entitled “Devices and Methods for the Delivery of Hemostatic Agents to Bleeding Wounds”; U.S. patent application Ser. No. 11/590,427, filed Oct. 30, 2006, entitled “Clay-Based Hemostatic Agents and Devices for the Delivery Thereof”; and U.S. patent application Attorney Docket No. 6989-0073, filed Nov. 29, 2006, entitled “Heat Mitigating Hemostatic Agent”; the contents of all of the above-referenced applications being incorporated herein by reference in their entireties.

### TECHNICAL FIELD

The present invention relates generally to agents and devices for promoting hemostasis and, more particularly, to silica-based hemostatic agents and devices incorporating such agents for the delivery thereof to bleeding wounds.

### BACKGROUND OF THE INVENTION

Blood is a liquid tissue that includes red cells, white cells, corpuscles, and platelets dispersed in a liquid phase. The liquid phase is plasma, which includes acids, lipids, solublized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances, animals, including humans, can be wounded. Often bleeding is associated with such wounds. In some circumstances, the wound and the bleeding are minor, and normal blood clotting functions in addition to the application of simple first aid are all that is required. Unfortunately, however, in other circumstances substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stanch the flow of blood in a timely manner.

Moreover, severe wounds can often be inflicted in remote areas or in situations, such as on a battlefield, where adequate medical assistance is not immediately available. In these instances, it is important to stop bleeding, even in less severe wounds, long enough to allow the injured person or animal to receive medical attention.

In an effort to address the above-described problems, materials have been developed for controlling excessive bleeding in situations where conventional aid is unavailable or less than optimally effective. Although these materials have been shown to be somewhat successful, they are sometimes not effective enough for traumatic wounds and tend to be expensive. Furthermore, these materials are sometimes ineffective in some situations and can be difficult to apply as well as remove from a wound.

Additionally, or alternatively, the previously developed materials can produce undesirable side effects. For example, one type of prior art blood clotting material is generally a powder or a fine particulate in which the surface area of the material often produces an exothermic reaction upon the application of the material to blood. Oftentimes excess material is unnecessarily poured onto a wound, which can exacerbate the exothermic effects. Depending upon the specific attributes of the material, the resulting exothermia may be sufficient to cause discomfort to or even burn the patient. Although some prior art patents specifically recite the resulting exothermia as being a desirable feature that can provide clotting effects to the wound that are similar to cauterization, there exists the possibility that the tissue at and around the wound site may be undesirably impacted.

Furthermore, to remove such materials from wounds, irrigation of the wound is often required. If an amount of material is administered that causes discomfort or burning, the wound may require immediate flushing. In instances where a wounded person or animal has not yet been transported to a facility capable of providing the needed irrigation, undesirable effects or over-treatment of the wound may result.

Bleeding can also be a problem during surgical procedures. Apart from suturing or stapling an incision or internally bleeding area, bleeding is often controlled using a sponge or other material used to exert pressure against the bleed site and/or absorb the blood. However, when the bleeding becomes excessive, these measures may not be sufficient to stop the blood flow. Moreover, any highly exothermic bleed-control material may damage the tissue surrounding the bleed site and may not be configured for easy removal after use.

Based on the foregoing, it is a general object of the present invention to provide a hemostatic agent that overcomes or improves upon the drawbacks associated with the prior art. It is also a general object of the present invention to provide devices capable of applying such hemostatic agents.

### SUMMARY OF THE INVENTION

According to one aspect, the present invention resides in a hemostatic agent comprising diatomaceous earth in particle form. When a bleeding wound is treated using such an agent, the application of at least a portion of the diatomaceous earth to the bleeding wound causes blood emanating from the wound to clot.

According to another aspect, the present invention resides in a device for promoting hemostasis. Such a device comprises diatomaceous earth in particle form and a receptacle for retaining the particles therein. At least a portion of the receptacle is defined by a mesh having openings therein. When treating a bleeding wound, application of the device causes at least a portion of the diatomaceous earth to come into contact with blood through the openings.

According to another aspect, the present invention resides in a hemostatic sponge comprising a substrate, diatomaceous earth disposed on a first surface of the substrate, and a release agent disposed on a second surface of the substrate. When this sponge is applied to a bleeding wound, blood comes into contact with the diatomaceous earth through the release agent and the substrate to cause a clotting effect.

According to another aspect, another embodiment of a hemostatic sponge comprises a film into which diatomaceous earth is incorporated. According to still another aspect, another embodiment of a hemostatic sponge comprises a substrate, diatomaceous earth disposed on the substrate, and a film disposed over the diatomaceous earth. According to still another aspect, an embodiment of a hemostatic sponge comprises a first substrate, diatomaceous earth disposed on the first substrate, and a second substrate disposed on the diatomaceous earth. When treating a bleeding wound using any of the foregoing sponge embodiments, application of the sponge causes at least a portion of the diatomaceous earth to come into contact with blood to cause a clotting effect.

According to other aspects, the present invention resides in devices that incorporate gauze substrates having diatomaceous earth, bandages incorporating diatomaceous earth, and methods of depositing diatomaceous earth onto gauze or other substrates. In any of the foregoing embodiments, application of the devices or bandages to bleeding wounds causes at least a portion of the diatomaceous earth to come into contact with blood, thereby causing clotting.

An advantage of the present invention is that unlike other materials, such as, for example zeolites, the diatomaceous earth produces no exothermic reaction with blood. Eliminating the generation of heat at a wound site is useful in minimizing discomfort and/or further injury to a patient and may be especially useful in the treatment of certain patients such as pediatric patients or when the wound being treated is in a particularly sensitive or delicate area.

Another advantage is that the diatomaceous earth or other silica-base material can be finely divided and deposited on a multitude of surfaces, thereby facilitating its use as a component in a variety of blood control devices. In particular, the diatomaceous earth can be used in particle form (e.g., retained in a mesh), or it can be used in powder form (e.g., deposited on a fibrous substrate to form a gauze or a sponge). In any embodiment, the efficacy of the diatomaceous earth in promoting hemostasis at a wound site is improved over similar agents that can be used only in one form (e.g., as particles of a particular size) to limit undesirable side effects such as excessive exothermic reactions.

Still another advantage of the present invention is that the devices and agents of the present invention are easily applied to open wounds. Particularly when the diatomaceous earth is retained in a mesh or similar device, or when it is incorporated into a woven structure to form a gauze, the device can be readily removed from a sterilized packaging and placed or held directly at the points from which blood emanates to cause clotting.

### BRIEF DESCRIPTION OF THE DRAWINGS

FIG. 1 is a schematic representation of a mesh structure of a hemostatic device of the present invention.

FIG. 2 is a side view of the hemostatic device of FIG. 1 illustrating the retaining of diatomaceous earth particles in the mesh structure.

FIG. 3 is a side view of a hemostatic device incorporating particles of a hemostatic agent in a mesh impregnated with particles of diatomaceous earth.

FIG. 4 is a side view of one embodiment of the mesh of the device of FIG. 3.

FIG. 5 is a side view of another embodiment of the mesh of the device of FIG. 3.

FIG. 6 is a side view of another embodiment of the mesh of the device of FIG. 3.

FIG. 7 is a perspective view of a hemostatic device that incorporates diatomaceous earth into a gauze.

FIG. 8 is a perspective view of a hemostatic device that incorporates diatomaceous earth into a cloth.

FIG. 9 is a perspective view of a bandage incorporating the diatomaceous earth particles in a mesh container for application to a bleeding wound.

FIG. 10 is a schematic representation of a sponge having hemostatic capabilities.

FIG. 11 is a schematic representation of another embodiment of a sponge having hemostatic capabilities.

FIG. 12 is a schematic representation of another embodiment of a sponge having hemostatic capabilities.

FIG. 13 is a schematic representation of another embodiment of a sponge having hemostatic capabilities.

### DETAILED DESCRIPTION OF THE PREFERRED EMBODIMENTS

Disclosed herein are hemostatic devices and hemostatic agents that are applicable to bleeding wounds to promote hemostasis. The hemostatic agents generally include diatomaceous earth that, when brought into contact with a bleeding wound, can minimize or stop blood flow by absorbing at least portions of the liquid phases of the blood, thereby facilitating clotting. The hemostatic devices and agents disclosed herein are not limited to diatomaceous earth, however, as the use of other silica-based materials such as clays in conjunction with diatomaceous earth is within the scope of the present invention.

As used herein, the term “diatomaceous earth” refers to a mineral derived from the fossilized shell remains of fresh water algae and marine algae. These algae are unicellular or colonial algae from the class Bacillariaphyccae and are known as diatoms. Diatoms are characterized by very irregular shapes and generally spiny structures having pitted surface areas. Structurally, they may be barrel-shaped, cylindrical, disk-shaped, etc. and average about 5 to about 20 microns in diameter.

The diatomaceous earth mineral, which is composed of the diatoms and is typically found in deposits in sedimentary rock formed as the result of receding waterlines in lakes and oceans, is about 86% silicon, about 5% sodium, about 3% magnesium, and about 2% iron, such components typically being present in oxide form. Other elements such as copper, strontium, manganese, titanium, and sodium, as well as other elements, may also be found in diatomaceous earth. The porosity of diatomaceous earth is about 85%.

For the hemostatic devices and agents of the present invention, various materials may be mixed with, associated with, or incorporated into the diatomaceous earth to maintain an antiseptic environment at the wound site or to provide other beneficial functions. Exemplary materials that can be used include, but are not limited to, antibiotics, antifungal agents, antimicrobial agents, anti-inflammatory agents, analgesics, antihistamines (e.g., cimetidine, chloropheniramine maleate, diphenhydramine hydrochloride, and promethazine hydrochloride), compounds containing silver or copper ions, combinations of the foregoing, and the like. Other materials that can be incorporated to provide additional hemostatic functions include ascorbic acid, tranexamic acid, rutin, thrombin, and combinations of the foregoing materials. Botanical agents having desirable effects on the wound site may also be added. The diatomaceous earth of the present invention may also be mixed with or otherwise used in conjunction with other materials to provide additional clotting functions and/or improved efficacy. Such materials include, but are not limited to, magnesium sulfate, sodium metaphosphate, calcium chloride, dextrin, combinations of the foregoing materials, and hydrates of the foregoing materials.

For use in the present invention, the diatomaceous earth is preferably formed into particles. As used herein, “particles” include beads, pellets, granules, rods, or any other surface morphology or combination of surface morphologies. Irrespective of the surface morphology, the particles are about 0.2 mm (millimeters) to about 10 mm, preferably about 0.5 mm to about 5 mm, and more preferably about 1 mm to about 2 mm in effective diameter. As used herein, the term “effective diameter” means the average diameter of the particle.

The diatomaceous earth is formed into particles by any of several various methods. Such methods include mixing, extrusion, spheronizing, and the like. Equipment that can be utilized for the mixing, extruding, or spheronizing of the diatomaceous earth is available from Caleva Process Solutions Ltd. in Dorset, United Kingdom. Other methods include the use of a fluid bed or a pelletizing apparatus. Fluid beds for the production of diatomaceous earth particles are available from Glatt Air Technologies in Ramsey, N.J. Disk pelletizers for the production of diatomaceous earth particles are available from Feeco International, Inc., in Green Bay, Wis. Preferably, the diatomaceous earth is extruded through a suitable pelletizing device. The present invention is not limited in this regard, however, as other devices and methods for producing particlized diatomaceous earth are within the scope of the present invention.

It is believed that the cellular clotting mechanisms of diatomaceous earth, as well as other silica-based materials, activate certain contact factors when applied to blood. More specifically, it is believed that such materials initiate one or more adsorption-type mechanisms by which water is removed from the liquid phases of blood to facilitate clotting functions.

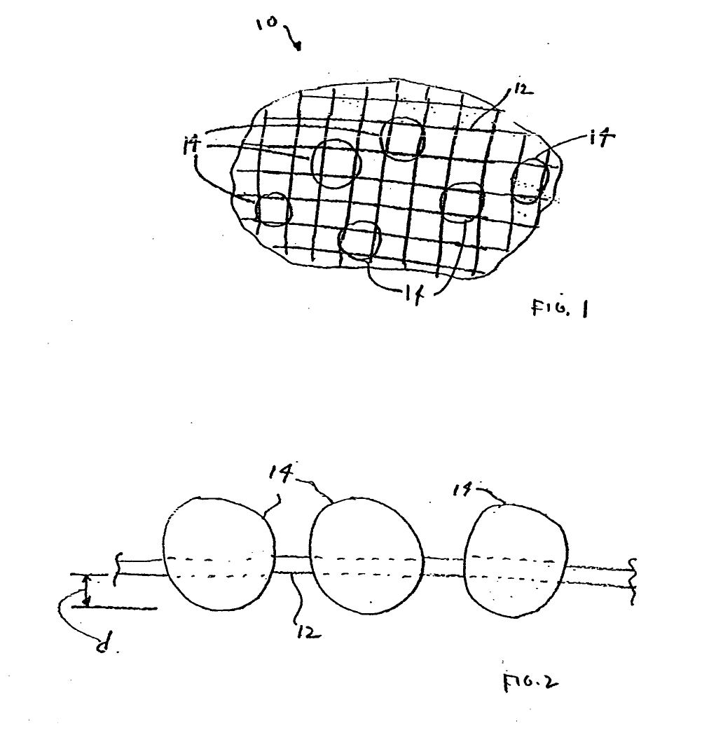

Referring now to FIG. 1, one embodiment of a hemostatic device into which the diatomaceous earth in particle form is incorporated is shown. The device is a permeable pouch that allows liquid to enter to contact the diatomaceous earth particles retained therein. Sealed packaging (not shown) provides a sterile environment for storing the hemostatic device until it can be used. The device, a portion of which is shown generally at 10 and is hereinafter referred to as “pouch 10,” comprises a screen or mesh 12 and the particlized diatomaceous earth 14 retained therein by the screen or mesh. The mesh 12 is closed on all sides and defines openings that are capable of retaining the particlized diatomaceous earth 14 therein while allowing liquid to flow through. As illustrated, the mesh 12 is shown as being flattened out, and, by way of example, only a few particles of particlized diatomaceous earth 14 are shown. The particlized diatomaceous earth 14 may be blended with particles of other materials such as clay, filler material, or the like to form a homogenous mixture.

The mesh 12 is defined by interconnected strands, filaments, or strips of material. The strands, filaments, or strips can be interconnected in any one or a combination of manners including, but not limited to, being woven into a gauze, intertwined, integrally-formed, and the like. Preferably, the interconnection is such that the mesh can flex while substantially maintaining the dimensions of the openings defined thereby. The material from which the strands, filaments or strips are fabricated may be a polymer (e.g., nylon, polyethylene, polypropylene, polyester, or the like), metal, fiberglass, or an organic substance (e.g., cotton, wool, silk, or the like).

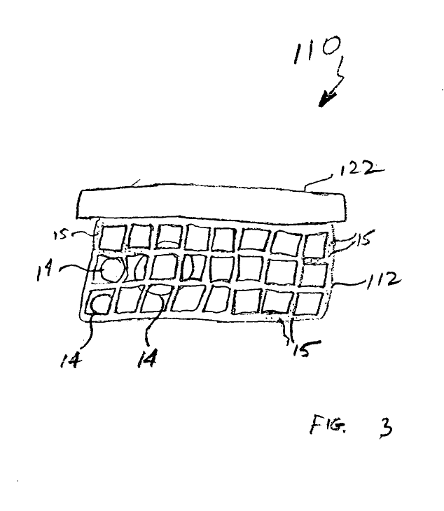

Referring now to FIG. 2, the openings defined by the mesh 12 are sized to retain the particlized diatomaceous earth 14 but permit the flow of blood therethrough. Because the mesh 12 may be pulled tight around the particlized diatomaceous earth 14, the particles may extend through the openings by a distance d. If the particles extend through the openings, they will directly contact tissue against which the pouch 10 is applied. Thus, blood emanating from the tissue immediately contacts the particlized diatomaceous earth 14, and the water phase thereof is wicked into the diatomaceous earth, thereby facilitating the clotting of the blood. However, it is not a requirement of the present invention that the particles protrude through the mesh.

To apply the pouch 10 to a bleeding wound, the pouch is removed from the packaging and placed on the bleeding wound. The particlized diatomaceous earth 14 in the mesh 12 contacts the tissue of the wound and/or the blood emanating from the wound, and at least a portion of the liquid phase of the blood is adsorbed by the clay material, thereby promoting clotting. The flexibility of the mesh 12 allows the mesh to conform to the shape of the bleeding wound and to retain that shape upon application.

Referring now to FIG. 3, another embodiment of the present invention comprises a device 110 having the diatomaceous earth particles as described above incorporated into a mesh 112, thereby providing hemostatic qualities to the material of the mesh itself. The mesh 112 is not limited to having diatomaceous earth, however, as other materials such as clay, bioglass, chitosan, oxidized cellulose, zeolite, or combinations of the foregoing may also be included in the mesh without deviating from the broader aspects of the present invention. The device 110 may include a support 122, thereby defining a pad. When the device 110 is a pad, the support 122 provides a surface at which pressure may be applied in the application of the device to a bleeding wound. Without the support 122, the device 110 may be used as a surgical sponge.

The diatomaceous earth-laden mesh 112 is defined by interconnected strands, filaments, or strips of material that are interconnected by being woven, intertwined, or integrally formed as in the above-disclosed embodiments. The mesh 112 includes particles 15 of diatomaceous earth. Although the particles 15 of diatomaceous earth are shown as being concentrated along portions of the edges of the mesh 112, it should be understood that the diatomaceous earth is dispersed throughout the material from which the mesh is fabricated. Preferably, the interconnection of the strands, filaments, or strips to form the mesh 112 is such that the device 110 can flex while substantially maintaining the dimensions of the openings, thereby allowing the diatomaceous earth (or other) particles 14 to be retained.

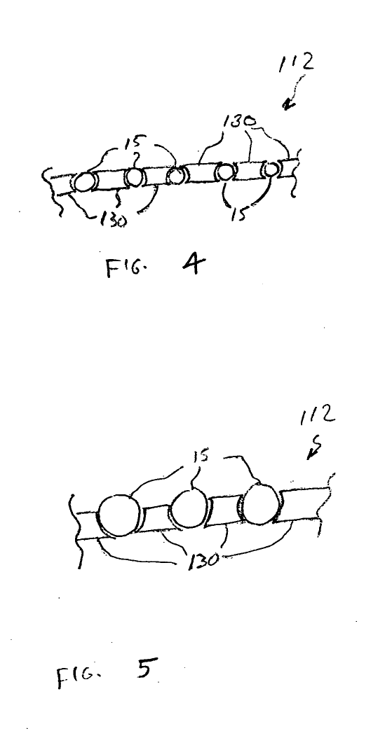

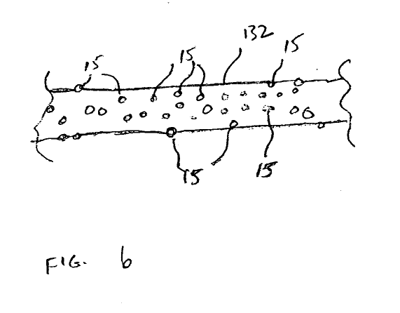

Referring now to FIGS. 4 and 5, diatomaceous earth is impregnated into or otherwise retained by the material of the strands, filaments, or strips that define the mesh 112. In particular, the particles 15 of diatomaceous earth may be captured within a matrix material 130 such that the particles contact the bleeding tissue when the strands, filaments, or strips defining the mesh 112 are brought into contact with the wound. As is shown in FIG. 4, the particles 15 of diatomaceous earth may be captured and held within the outer surface of the matrix material 130. In such an embodiment, the matrix material 130 is preferably sufficiently porous to facilitate the flow of blood therethrough, thus allowing liquid phases of the blood to be at least partially absorbed by the particles 15 prior to contacting the diatomaceous earth (or other material) retained in the mesh 112. As is shown in FIG. 5, the particles 15 of diatomaceous earth may be captured so as to protrude above the surface of the matrix material 130.

Referring to FIG. 6, the particles 15 of diatomaceous earth may be impregnated into a substrate material 132 of the mesh and retained therein by any suitable method. In the impregnation of the diatomaceous earth into the substrate material 132, the substrate material is generally sufficiently soft (e.g., fluid when exposed to heat) to allow for its deformation to accommodate the diatomaceous earth. The particles 15 may be impregnated completely into the substrate material 132, or it may be partially impregnated so as to extend out of the substrate material.

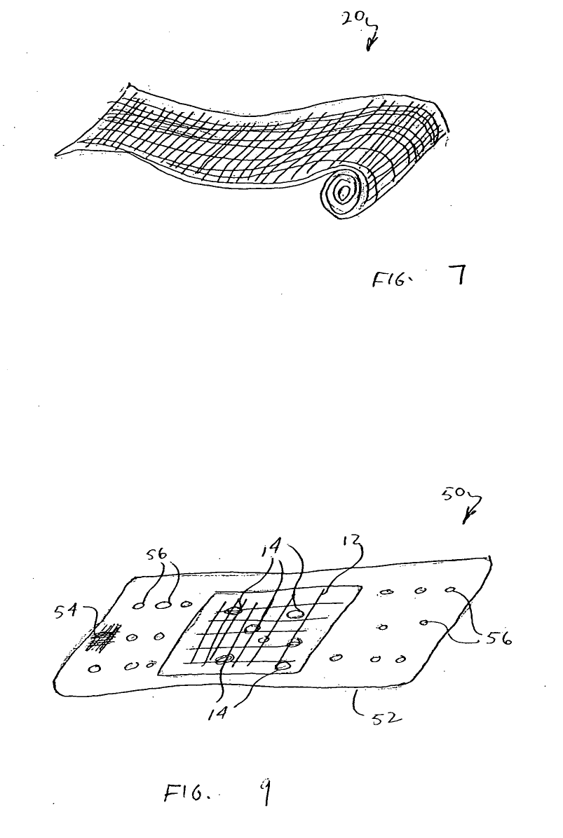

Referring now to FIG. 7, another embodiment of a hemostatic device of the present invention is a gauze that incorporates diatomaceous earth, which is shown generally at 20 and is hereinafter referred to as “gauze 20.” Diatomaceous earth is coated onto a gauze substrate using any suitable method. One exemplary method of coating diatomaceous earth onto the gauze substrate is to immerse the substrate in a slurry of diatomaceous earth and water. The diatomaceous earth used for the slurry is preferably finely ground powder, although the present invention is not limited in this regard as particles, flakes, chips, beads, rods, granules, or the like may alternatively or additionally be used. The gauze substrate may be any suitable woven or non-woven fibrous material including, but not limited to, cotton, silk, wool, plastic, cellulose, rayon, polyester, combinations of the foregoing, and the like. The present invention is not limited to woven and non-woven fibrous materials as the gauze substrates, however, as felts and the like are also within the scope of the present invention.

The gauze 20 of the present invention is not limited solely to diatomaceous earth, however, as clays such as attapulgite, bentonite, and combinations thereof may be used in addition to the diatomaceous earth. Furthermore, other silica-based materials such as bioactive glasses and the like may also be utilized in addition to diatomaceous earth.

In any embodiment, once the diatomaceous earth is dried onto the gauze substrate to form the gauze 20, the gauze is sufficiently flexible to allow the gauze to be folded, rolled, or otherwise manipulated for packaging. The flexibility of the substrate of the gauze 20 allows the gauze to form to a shape of the bleeding wound and to retain the shape of the bleeding wound upon application.

One manner of causing the diatomaceous earth coating to be retained on the gauze substrate includes combining the diatomaceous earth with a binder such as chitosan. The present invention is not limited to the use of chitosan as a binder however, as other materials (e.g., polysaccharides, polyvinyl alcohol, guar gum, glycerol, gelatinized starches, cellulose (e.g., carboxymethyl cellulose), calcium alginate, and the like) are suitable for use as binders and therefore within the scope of the present invention. In any embodiment, the material of the binder is biocompatible.

Other manners of causing the diatomaceous earth to be retained on the gauze include the use of heat. More specifically, it has been found that by heating a diatomaceous earth/water slurry to or near boiling temperatures, the adhesion of the diatomaceous earth to the gauze material is facilitated. Preferably, the slurry is heated to boiling because higher temperatures tend to promote the adhesion of the diatomaceous earth to the gauze while also providing an effective form of agitation that uniformly disperses the diatomaceous earth in the liquid phase. The gauze (or other substrate material) is then immersed in the boiling slurry for an amount of time sufficient to cause the diatomaceous earth to deposit onto the gauze material. Given the rheology of wetted diatomaceous earth and the material from which the gauze or substrate is fabricated, the diatomaceous earth may adhere as a film directly to the surfaces of the substrate, or it may agglomerate in the interstices of the strands as well as along the strands themselves, thereby being trapped in the fiber matrix.

Another manner of depositing the kaolin coating on the substrate includes applying the diatomaceous earth in slurry form on one side of the gauze substrate using a spraying technique, a slot die technique, or a combination thereof. In using any technique, the amount of slurry applied to the gauze substrate is limited to avoid or at least minimize the saturation of the substrate. Preferably, the diatomaceous earth provides a stable suspension of the material with suitable viscosity for application using the slot die technique.

Once sprayed or applied using the slot die technique, the coated gauze substrate is then rolled or scraped to further embed the diatomaceous earth into the material of the substrate. The gauze substrate is then dried.

In some embodiments, the diatomaceous earth may be combined with the binder and sprayed onto the gauze substrate. As indicated above, preferred binders include chitosan or other polysaccharides as well as polyvinyl alcohol, all of which have adhesive qualities, are compatible with biological tissue, and also exhibit hemostatic properties.

One exemplary method for the production of this device may comprise the steps of unwinding cotton gauze from a roll, immersing the gauze in a slurry of diatomaceous earth and water, applying pressure to the gauze by rolling the wet gauze under high pressure to incorporate the hemostatic material into the material of the gauze, drying the rolled, wet gauze, and removing dust from the gauze (e.g., via blasting with air knives or air nozzles, through the use of electrostatic energy, vacuuming, or brushing with direct contact brushes). Following the removal of dust from the gauze, the gauze may be wound back onto a roll, or it may be cut into sheets for individual packaging.

One or more variables may be manipulated to optimize the amount and integrity of the diatomaceous earth retained on the gauze. These variables include, but are not limited to, slurry temperature, immersion time, the slurry agitation method, and the type of liquid (of the slurry). The elevation of the slurry temperature, as indicated above, aids in the retention of the diatomaceous earth on the gauze. The agitation may be effected by forcing air or other gas through nozzles, stirring, bubbling, boiling, or ultrasonic vibration.

The liquid used for the slurry may also be something other than water. For example, the liquid may be an aqueous ammonia solution. Aqueous ammonia has been found to induce swelling in certain fibrous materials, such as the materials typically utilized to fabricate gauze.

Referring now to FIG. 8, another embodiment of a hemostatic device of the present invention is a cloth having hemostatic properties, shown generally at 30, and which is hereinafter referred to as “cloth 30.” The cloth 30 is a fabric which may be defined by woven or unwoven strands or a felt or the like into which diatomaceous earth, shown at 32, is infused or impregnated.

In either gauze or cloth embodiments, the gauze or cloth material may be cross-linked with a polysaccharide or similar material.

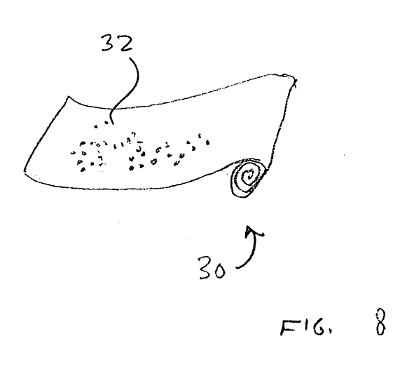

Referring now to FIG. 9, another embodiment of the present invention is a bandage, shown at 50, which comprises particlized diatomaceous earth 14 retained in the mesh 12 and mounted to a flexible substrate 52 that can be applied to a wound (for example, using a pressure-sensitive adhesive to adhere the bandage 50 to the skin of a wearer). The mesh 12 is stitched, glued, or otherwise mounted to the substrate 52 to form the bandage 50.

The substrate 52 is a plastic or a cloth member that is conducive to being retained on the skin of an injured person or animal on or proximate a bleeding wound. An adhesive 54 is disposed on a surface of the substrate 52 that engages the skin of the injured person or animal. Particularly if the substrate 52 is a non-breathable plastic material, the substrate may include holes 56 to allow for the dissipation of moisture evaporating from the skin surface.

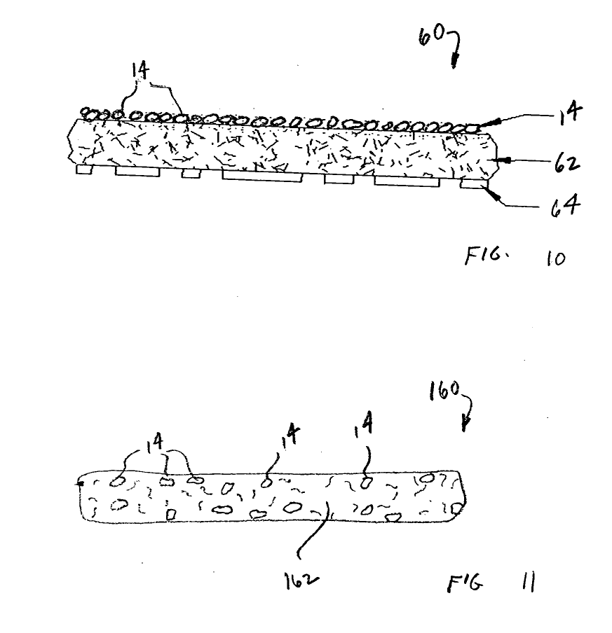

Referring now to FIG. 10, another embodiment of the present invention is a sponge, shown at 60, which comprises a substrate 62, the particlized diatomaceous earth 14 disposed on one face of the substrate 62, and a release agent 64 disposed on an opposing face of the substrate. The sponge 60 allows for sufficient contact of the particlized diatomaceous earth 14 with blood emanating from a wound and through the release agent 64 and the substrate 62 while minimizing the adhesion of the sponge to the wound tissue. The sponge 60 is also compatible with living tissue.

The substrate 62 is an absorbent gauze material that defines a matrix. The gauze may be defined as a rayon/polyester cellulose blend or the like. Other materials from which the substrate 62 may be fabricated include woven fabric, non-woven fabric, silk, paper (e.g., kraft paper and the like), and cellulose material (e.g., cotton in the forms of balls, swabs, and the like). Any material from which the substrate 62 may be fabricated may have an elastic quality. When elastic materials are used as the substrate 62, the sponge 60 becomes both a hemostatic device and a pressure bandage, particularly in embodiments in which a surface cohesive agent or mechanical fastener is added to secure the sponge in place over a wound.

The hemostatic agent used in the sponge 60 is not limited to particlized diatomaceous earth 14. Clay materials such as attapulgite, bentonite, or combinations of the foregoing may be used with diatomaceous earth. Furthermore, other materials such as bioactive glass, biological hemostats, and combinations thereof with diatomaceous earth are also within the scope of the present invention.

The particlized diatomaceous earth 14 may be bound to the substrate 62 via coulombic forces, by impregnating or otherwise incorporating the diatomaceous earth directly into the material of the substrate, by using a binder, by trapping the hemostatic material within the matrix, or the like.

When using a binder to bind the particlized diatomaceous earth 14 to the substrate 62, the binder material may provide additional functionality to the sponge 60. Materials from which the binder may be fabricated include, but are not limited to, chitosan, polyvinyl alcohol, guar gum, gelatinized starches, polysaccharides, glycerol, cellulose (e.g., carboxymethyl cellulose), calcium alginate, and the like, as well as combinations of the foregoing.

In embodiments in which the particlized diatomaceous earth 14 is incorporated into the substrate 62 directly, the particlized diatomaceous earth may be added during the substrate fabrication. If the substrate is a non-woven gauze material containing rayon and polyester, then the particlized diatomaceous earth 14 may be incorporated into or onto the fibers of rayon and polyester. For example, the particlized diatomaceous earth 14 may be in powder form and applied to molten polyester, and polyester fibers may be drawn from the polyester/diatomaceous earth melt. If the substrate is a woven gauze (e.g., cotton), the diatomaceous earth 14 in powder form may be incorporated into the cotton threads during formation of the threads.

The release agent 64 is a material that is disposed on the wound-contacting side of the substrate 62 to facilitate the easy removal of the sponge 60 from the wound tissue after the formation of blood clots. The release agent 64 may be a continuous film, or it may be discontinuous on the surface of the substrate. One material that may be used as a release agent is polyvinyl alcohol, which is a biocompatible material that may be formed as a thin film and that does not significantly affect the absorbency and liquid permeability of the sponge 60. The release agent 64 may be applied directly to the wound-contacting surface of the substrate 62.

In the alternative, the release agent 64 may be applied to the non-wound contacting surface of the substrate 62 as a slurry of diatomaceous earth and release agent. In such an embodiment, the concentration of the polyvinyl alcohol is such that at least some of the alcohol seeps to the wound-contacting surface of the substrate 62, while the diatomaceous earth remains on or near the non-wound contacting surface. In any embodiment, the polyvinyl alcohol serves not only as a release agent, but as an agent that suppresses the dust of the particlized diatomaceous earth 14.

Other materials that may be used as release agents that are within the scope of the present invention include, but are not limited to, silicone, glycerol, and gelatinized starches. As with polyvinyl alcohol, either may be applied in film form.

Referring now to FIG. 11, another embodiment of a sponge is shown generally at 160. The sponge 160 comprises a film 162 into which particlized diatomaceous earth 14 is dispersed. The physical integrity of the sponge 160 is maintained by the film 162. Preferably, the material from which the film 162 is fabricated is polyvinyl alcohol. In fabricating the sponge 160, the particlized diatomaceous earth 14 is dispersed into polyvinyl alcohol, which is then formed into a sheet. The sponge 160 is especially useful when incorporated into a bandage.

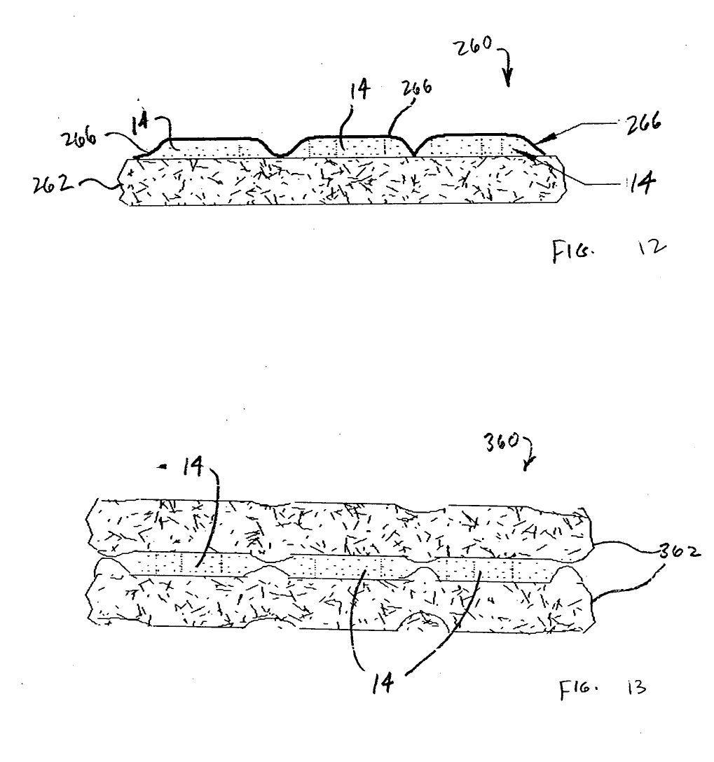

Referring now to FIG. 12, another embodiment of a sponge is shown generally at 260. The sponge 260 comprises a substrate 262, particlized diatomaceous earth 14 disposed on the substrate, and a film 266 disposed over the hemostatic material. The particlized diatomaceous earth 14 is unbound (without a binder) and is preferably disposed on the substrate 262 in strips to facilitate the folding of the sponge 260. The film 266 is polyvinyl alcohol or the like and is applied to both contain the particlized diatomaceous earth 14 and to minimize the generation of dust. Upon application to a bleeding wound, blood from the wound is wicked into the substrate 262 and contacts the particlized diatomaceous earth 14.

Referring now to FIG. 13, another embodiment of a sponge is shown generally at 360. The sponge 360 comprises particlized diatomaceous earth 14 sandwiched between two substrates 362. The substrates 362 can be bound together in any suitable manner such as heat sealing through areas selectively absent of particlized diatomaceous earth 14, using an adhesive or binder in select areas, applying a containment film of material (such as polyvinyl alcohol) over the entire sponge 360, or a combination of any of the foregoing.

The sponge 60 (as well as the sponges shown at 160, 260, and 360) may be folded and used in various manners. The sponge 60 may be folded such that the surfaces on which the particlized diatomaceous earth 14 is disposed are on the inside surfaces of the folded sponge, so as to minimize problems of dusting and detachment of the hemostatic material from the substrate 62. The sponge 60 (and the sponges 160, 260, and 360) can also be folded into a pleated form or into a configuration to produce a number of distinct plies attached along the edges. By configuring the sponge 60 in such a manner, the compliancy and absorbency requirements of different applications can be addressed. The sponge 60 can also be cut or formed into elongated strips for wrapping over the wounds of an injured person or animal or for incorporation into cylinders or swabs. The sponge 60 can also be cut, ripped, ground, or otherwise formed into small pieces for applications such as stuffing into mesh containers.

Also, the sponge 60 (as well as the sponges shown at 160, 260, and 360) may further include a component that imparts a radiopaque characteristic to the sponge. In such an embodiment, barium sulfate may be incorporated into a slurry that includes the particlized diatomaceous earth 14 and applied to the substrate 62, or the barium sulfate may be incorporated directly into the substrate material. Furthermore, the sponge 60 may further include water or alcohol incorporated into one or more of the substrate, the diatomaceous earth, and the release agent, thereby allowing the sponge to be used as a wipe.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A hemostatic agent, comprising: diatomaceous earth in particle form, wherein when treating a bleeding wound, application of at least a portion of said diatomaceous earth in particle form to said bleeding wound causes blood emanating from said bleeding wound to clot.

2. The hemostatic agent of claim 1, further comprising a material selected from the group consisting of antibiotics, antifungal agents, antimicrobial agents, anti-inflammatory agents, analgesics, antihistamines, compounds containing silver ions, compounds containing copper ions, and combinations of the foregoing compositions.

3. The hemostatic agent of claim 1, further comprising a material selected from the group consisting of magnesium sulfate, sodium metaphosphate, calcium chloride, dextrin, hydrates of the foregoing materials, and combinations of the foregoing materials.

4. The hemostatic agent of claim 1, further comprising a material selected from the group consisting of ascorbic acid, tranexamic acid, rutin, thrombin, and combinations of the foregoing materials.

5. The hemostatic agent of claim 1, wherein said diatomaceous earth comprises particles having diameters of about 0.2 mm to about 10 mm.

6. The hemostatic agent of claim 1, wherein said diatomaceous earth comprises particles having diameters of about 1 mm to about 7 mm.

7. The hemostatic agent of claim 1, wherein said diatomaceous earth comprises particles having diameters of about 2 mm to about 5 mm.

8. A device for promoting hemostasis, comprising:
diatomaceous earth in particle form;
a receptacle for retaining said diatomaceous earth therein, at least a portion of said receptacle being defined by a mesh having openings therein;
wherein when treating a bleeding wound, application of said device causes at least a portion of said diatomaceous earth to come into contact with blood through said openings.

9. The device for promoting hemostasis of claim 8, wherein said mesh is flexible.

10. The device for promoting hemostasis of claim 9, wherein the flexibility of said mesh allows said mesh to form to a shape of said bleeding wound and to retain a shape of said bleeding wound.

11. The device for promoting hemostasis of claim 8, wherein at least one particle of said diatomaceous earth protrudes through one of said openings.

12. A device capable of providing a hemostatic effect on a bleeding wound, said device comprising:
a gauze substrate; and
diatomaceous earth disposed on said gauze substrate;
wherein when treating a bleeding wound, application of said device causes at least a portion of said diatomaceous earth to come into contact with blood.

13. The device of claim 12, further comprising a clay material selected from the group consisting of attapulgite, bentonite, kaolin, and combinations of the foregoing materials.

14. The device of claim 12, wherein said gauze substrate is fabricated from a material selected from the group consisting of cotton, silk, wool, plastic, cellulose, rayon, polyester, and combinations of the foregoing.

15. The device of claim 12, wherein said gauze substrate is flexible to allow said gauze substrate to form to a shape of said bleeding wound and to retain a shape of said bleeding wound.

16. The device of claim 12, further comprising a binder to adhere said diatomaceous earth to said gauze substrate.

17. The device of claim 16, wherein said binder is selected from the group consisting of chitosan, polysaccharides, guar gum, gelatinized starches, glycerol, cellulose, calcium alginate, and polyvinyl alcohol.

18. A bandage applicable to a bleeding wound, said bandage comprising:
a substrate;
a mesh mounted on said substrate; and
diatomaceous earth in particle form retained in said mesh;
said mesh defined by a plurality of members arranged to define openings, said openings being dimensioned to accommodate the flow of blood therethrough.

19. The bandage of claim 18, further comprising an adhesive on said substrate, said adhesive being configured to facilitate the retaining of said bandage on the skin of a wearer.

20. The bandage of claim 18, wherein said substrate includes holes.

21. A method for producing a hemostatic gauze, said method comprising the steps of:
unwinding a gauze from a first roll; and
depositing a slurry comprising diatomaceous earth and water on said gauze.

22. The method of claim 21, wherein said step of depositing said slurry of diatomaceous earth and water on said gauze comprises immersing said gauze into said slurry.

23. The method of claim 21, wherein said step of depositing said slurry of diatomaceous earth and water on said gauze comprises utilizing a slot die technique.

24. The method of claim 21, wherein said step of depositing said slurry of diatomaceous earth and water on said gauze comprises spraying said slurry onto said gauze.

25. The method of claim 21, further comprising incorporating said diatomaceous earth into said gauze by applying pressure to said wet gauze.

26. The method of claim 21, further comprising removing dust from said gauze.

27. The method of claim 21, further comprising winding said gauze incorporating said diatomaceous earth into a second roll.

28. The method of claim 21, further comprising cutting said gauze into sheets.

29. The method of claim 26, wherein said step of removing dust from said gauze is selected from the group consisting of blasting said gauze with air, applying electrostatic energy to said gauze, vacuuming said gauze, and brushing said gauze.

30. The method of claim 21, wherein said slurry comprising diatomaceous earth and water further comprises a binder.

31. A hemostatic sponge, comprising:
a substrate;
diatomaceous earth disposed on a first surface of said substrate; and
a release agent disposed on a second surface of said substrate;
wherein when treating a bleeding wound, application of said hemostatic sponge causes at least a portion of said diatomaceous earth to come into contact with blood through said release agent and said substrate.

32. The hemostatic sponge of claim 31, wherein said substrate is a material selected from the group consisting of cotton, rayon/polyester cellulose blends, cellulose, paper, and combinations of the foregoing.

33. The hemostatic sponge of claim 31, further comprising a binder to adhere said diatomaceous earth to said substrate.

34. The hemostatic sponge of claim 33, wherein said binder is selected from the group consisting of chitosan, polysaccharides, guar gum, gelatinized starches, glycerol, cellulose, calcium alginate, and polyvinyl alcohol.

35. The hemostatic sponge of claim 31, wherein said release agent is selected from the group consisting of polyvinyl alcohol, silicone, glycerol, and gelatinized starch.

36. The hemostatic sponge of claim 31, further comprising at least one of water and alcohol incorporated into said substrate.

37. The hemostatic sponge of claim 31, further comprising a radiopaque agent incorporated into at least one of said substrate, said diatomaceous earth, and said release agent.

38. A hemostatic sponge, comprising:
a film; and
diatomaceous earth incorporated into said film;
wherein when treating a bleeding wound, application of said hemostatic sponge causes at least a portion of said diatomaceous earth to come into contact with blood emanating from said bleeding wound.

39. The hemostatic sponge of claim 38, wherein said film is polyvinyl alcohol.

40. A hemostatic sponge, comprising:
a substrate;
diatomaceous earth disposed on said substrate; and
a film disposed over said diatomaceous earth;
wherein when treating a bleeding wound, application of said hemostatic sponge causes at least a portion of said diatomaceous earth to come into contact with blood through said substrate.

41. The hemostatic sponge of claim 40, wherein said diatomaceous earth is disposed on said substrate in strips to facilitate the subsequent heat sealing or ultrasonic sealing of said hemostatic sponge.

42. The hemostatic sponge of claim 40, wherein said hemostatic sponge further comprises barium sulfate incorporated into at least one of said substrate, said diatomaceous earth, and said film.

43. A hemostatic sponge, comprising:
a first substrate;
diatomaceous earth disposed on said first substrate; and
a second substrate disposed on said diatomaceous earth;
wherein when treating a bleeding wound, application of said hemostatic sponge causes at least a portion of said diatomaceous earth to come into contact with blood through at least one of said first substrate and said second substrate.

44. The hemostatic sponge of claim 43, wherein said hemostatic sponge further comprises barium sulfate incorporated into at least one of said first substrate, said diatomaceous earth, and said second substrate.
